(19) United States
(12) Patent Application Publication
(10) Pub. No.: US 2018/0008333 A1
(43) Pub. Date: Jan. 11, 2018

(54) SKIN CARE APPARATUS USING PLASMA AND NEAR INFRARED RAY

(71) Applicant: Seok JIN, Seoul (KR)

(72) Inventor: Seok JIN, Seoul (KR)

(21) Appl. No.: 15/293,141

(22) Filed: Oct. 13, 2016

(30) Foreign Application Priority Data
Jul. 8, 2016 (KR) 10-2016-0086713

(51) Int. Cl. A61B 18/04; A61N 5/06; A61N 1/44; A61B 18/00

(52) U.S. Cl. CPC A61B 18/042; A61N 1/44; A61N 5/0616; A61N 2005/0644; A61B 2018/0016; A61N 2005/0654; A61N 2005/0659; A61N 2005/0626

(57) ABSTRACT

An exemplary embodiments of the present disclosure are directed to providing a skin care apparatus using plasma and near-infrared ray designed to provide effects of improving skin and lifting by irradiating plasma and near-infrared ray generated through dual electric generation on skin contacted by a spherical form. Further, the exemplary embodiments of the present disclosures are directed to providing a skin care apparatus using plasma and near-infrared ray that prevents skin contact or dual irradiation, which were used to happen in the conventional side contact structure, with the method of irradiating plasma and near-infrared ray by using dielectric barrier discharge for each of the plasma discharge groove and near-infrared irradiation groove that are different from the conventional contact structure, through a spot contact spherical body structure.

### CROSS-REFERENCE TO RELATED APPLICATIONS

This application claims the benefit under 35 USC 119(a) of Korean Patent Application No. 10-2016-00086713 filed on Jul. 8, 2016 in the Korean Intellectual Property Office, the entire disclosure of which is incorporated herein by reference for all purposes.

### BACKGROUND

### 1. Field

The present disclosure relates to a skin beauty apparatus. More particularly, the present disclosure relates to a skin beauty apparatus designed to provide effects of improving skin and lifting by irradiating plasma and near-infrared ray generated through dual electric generation on skin contacted by a spherical form.

### 2. Description of Related Art

The anti-aging market is growing recently. Anti-aging signifies various treatments designed to medically deal with aging process and to help keeping youth. The demand for the anti-aging market is increasing due to the aging phenomenon, the tendency to lay stress on appearances, the increase of the income, the increased entry of women in the society.

Further, the skin beauty apparatus market is growing fast. The paradigm of anti-aging industry has been transformed from the past cosmetic-centered market to the high-value market. Therefore, it is estimated that the growth of the skin beauty apparatus market will be accelerated along with the growth of the face beauty market as the market is expected to be reorganized around cosmetic procedures as its center.

Accordingly, the home beauty care apparatus market is growing.

As the development of home beauty care apparatus increases, it is expected that a fair number of cosmetic procedures now performed at a dermatologist will be performed at home. The beauty care apparatuses are the apparatuses for the skin management and skin protection and customer needs for them are increasing. As the need for cosmetic procedures increases, beauty care apparatus such as one using laser for hospital has emerged. However, as customers still feel economically burdened and feel temporal pressure about going to hospital for cosmetic procedures, the demand for home beauty care apparatus is increasing. The home beauty care apparatus has both characteristics of consumer goods business and medical appliances and the home beauty care apparatus market is expected to expand later with the development of personalized health care service.

Meanwhile, the population of women is gradually changing according to the change in the social environment. The development of the skin care industry is related to the increase in the entry of women in the society and increase of their income. As the women's social status has changed and using beauty care apparatus and service is considered to be self-investment, the skin care market is growing. This trend will continue in the future.

Next, the trend is changing from “well-being” to “wellness”. The characteristic of “wellness” is that people want to look younger compared to the same age group and to have a healthy body. With such change in trend, the anti-aging industry targeted for youth and beauty is rapidly growing. Particularly, skin condition is an index of age and health. Therefore, as keeping a good skin condition is considered to be a key to anti-aging, the growth in related industry including medical procedures, functional cosmetics, and beauty services is expected.

Meanwhile, the conventional skin beauty apparatuses are apparatuses using laser or using high intensive focused ultrasound.

First, the skin beauty apparatus using laser utilizes photon to cure tissues. These photons work on tissues by being reflected or refracting on them or by being integrated into chromophores. Various wavelengths and mediums are used for the purpose of appropriate function of it.

However, the skin beauty apparatus using laser may damage the optic nerve system or the base plate of epidermal cells. Further, this skin beauty apparatus may cause infection or necrosis.

Further, the skin beauty apparatus using High Intensity Focused Ultrasound performs a non-invasive procedure that does not damage skin. This skin beauty apparatus does not cause bleeding after procedures. However, as this skin beauty apparatus reforms collagen by forming a solidification point between 1.5 mm and 4.5 mm, the treating effects may be limited to lifting.

Meanwhile, skin beauty apparatuses using plasma or micro current instead of laser and ultrasonic wave are being developed.

The skin beauty apparatus that transforms moisture and oxygen into plasma and accelerates the formation of collagen by interfusing it to the skin in order to ameliorate wrinkles and improve skin elasticity is being developed.

The skin beauty apparatus using plasma enables skin touch by allowing constant contact area and functions. However, it is possible that plasma can be doubly irradiated by discharging of random area of plasma and rubbing in this skin beauty apparatus. Further, it has a disadvantage that it may be inconvenient in narrow regions because of its constant contact area.

Also, a skin beauty apparatus that enables the face or whole body rolling with a rotation roller by generating micro current is being developed.

The skin beauty apparatus using micro current pursues skin metabolism activation, just using the micro current. The effect of massage is a main function due to the adhesion structure between two spherical forms. It may supremely work on wide bodies such as cheeks or forehead, but it may cause inconveniences on narrow parts.

### SUMMARY

The exemplary embodiments of the present disclosure are directed to providing a skin care apparatus using plasma and near-infrared ray designed to provide effects of improving skin and lifting by irradiating plasma and near-infrared ray generated through dual electric generation on skin contacted by a spherical form.

Further, the exemplary embodiments of the present disclosures are directed to providing a skin care apparatus using plasma and near-infrared ray that prevents skin contact or dual irradiation, which were used to happen in the conventional side contact structure, with the method of irradiating plasma and near-infrared ray by using dielectric barrier discharge for each of the plasma discharge groove and near-infrared irradiation groove that are different from the conventional contact structure, through a spot contact spherical body structure.

Further, the exemplary embodiments of the present disclosure are directed to providing a skin care apparatus using plasma and near-infrared ray that can be used as a home beauty care device designed to ameliorate wrinkles and improve skin elasticity through a dual electric generation of plasma and near-infrared ray.

According to the first aspect of the present disclosure, a skin care apparatus using plasma and near-infrared ray can be provided that includes a main body in the form of handheld; a spherical body connected with a rotational axis of the main body to be rotatable, and the spherical body having a plurality of plasma discharge grooves and a plurality of near-infrared irradiation grooves formed on a surface thereof; a plasma discharger configured to comprise a dielectric plate and an electrode respectively corresponding to the plasma discharge grooves and discharge plasma generated through dielectric barrier discharge caused by a current supplied to the dielectric plate and electrode on skin contacted by the spherical body being rotating; a near-infrared irradiation unit configured to comprise a near-infrared light source linked with the near-infrared irradiation groove and irradiates a near-infrared ray generated from the near-infrared light source through the near-infrared irradiation groove on the skin contacted by the spherical body being rotating; a control unit configured to control a plasma discharge at the plasma discharger and an irradiation of near-infrared ray at the near-infrared irradiation unit; and a power supply configured to supply power.

The spherical body comprises at least one pair of spherical bodies, having a plurality of plasma discharge grooves formed on the surface thereof, the plurality of plasma discharge grooves having a depth conforming to the predetermined generation condition of plasma.

The spherical body further comprises at least one other spherical body, being located between the at least one pair of spherical bodies connected with the rotational axis of the main body to be rotatable, and has a plurality of plasma discharge grooves and a plurality of near-infrared irradiation grooves formed thereon.

The surface of the spherical body is divided into a plurality of boundary layers and a plurality of plasma discharge grooves being formed on each of the boundary layers.

The part of the spherical body connected to the rotational axis of the spherical body is made of an angle adjustable elastic material or by an articulated structure.

The control unit controls a plasma pulse and near-infrared ray pulse or a output of plasma and near-infrared ray according to a pulse pattern or output pattern established for a user skin type.

The control unit comprises a plasma pulse control unit configured to control the plasma pulse according to the pulse pattern established for the user skin type; a near-infrared pulse control unit configured to control near-infrared ray pulse according to the pulse pattern established for the user skin type; and a plasma and near-infrared ray output control unit configured to control the output of plasma and near-infrared ray according to the output pattern established for the user skin type.

The above apparatus comprises a user interface unit configured to receive a user input for plasma discharge or near-infrared ray irradiation and display the results of the user input.

The exemplary embodiments of the present disclosure provide the effects of skin improvement and lifting by irradiating plasma and near-infrared ray generated by the dual electric generation on the skin contacted by the rotatable spherical body.

Further, the exemplary embodiments of the present disclosure prevents skin contact or redundant irradiation that used to happen in the plain contact structure by face rolling since they irradiate plasma and near-infrared ray on each of the plasma discharge grooves and near-infrared ray grooves by using dielectric barrier discharge in the spot contacting spherical body.

Further, the exemplary embodiments of the present disclosure can be used as a home beauty care device that ameliorates wrinkles and improve skin elasticity through the dual electric generation of plasma and near-infrared ray.

### BRIEF DESCRIPTION OF THE DRAWINGS

FIG. 1 is a diagram illustrating a skin care apparatus using plasma and near-infrared ray according to an exemplary embodiment of the present disclosure.

FIG. 2 is a drawing illustrating the lower side, the side, and the upper side of a skin care apparatus using plasma and near-infrared ray according to an exemplary embodiment of the present disclosure.

FIG. 3 is a drawing illustrating the system of plasma discharge and irradiation of infrared ray at a spherical body according to an exemplary embodiment of the present disclosure.

FIG. 4 is an end view of the inside of a spherical body according to an exemplary embodiment of the present disclosure.

FIG. 5 is a structural drawing illustrating a dielectric plate and an electrode of a plasma discharge unit according to an exemplary embodiment of the present disclosure.

FIG. 6 is a graph showing a pulse width modulation control waveform controlled by a control unit according to an exemplary embodiment of the present disclosure.

FIG. 7 is a drawing illustrating a roller action of a skin care apparatus using plasma and near-infrared ray according to an exemplary embodiment of the present disclosure.

### DETAILED DESCRIPTION

The present disclosure will be described more fully hereinafter with reference to the accompanying drawings, in which exemplary embodiments of the disclosure are shown. The description of technology generally known to those skilled in the art will be omitted to clearly transfer the spirit of the present disclosure. Also, well-known functions or constructions are not described in detail to avoid obscuring the examples with unnecessary detail.

Further, in the following description, the same drawing reference numerals are used for the same elements, even in different drawings. Also, different drawing reference numerals may be used for the same elements depending on the drawings. The matters defined in the description, such as detailed constructions of terms and elements, are provided to assist in a comprehensive understanding of the present examples. Accordingly, it is apparent that it is possible for the examples to be carried out without those specifically defined matters.

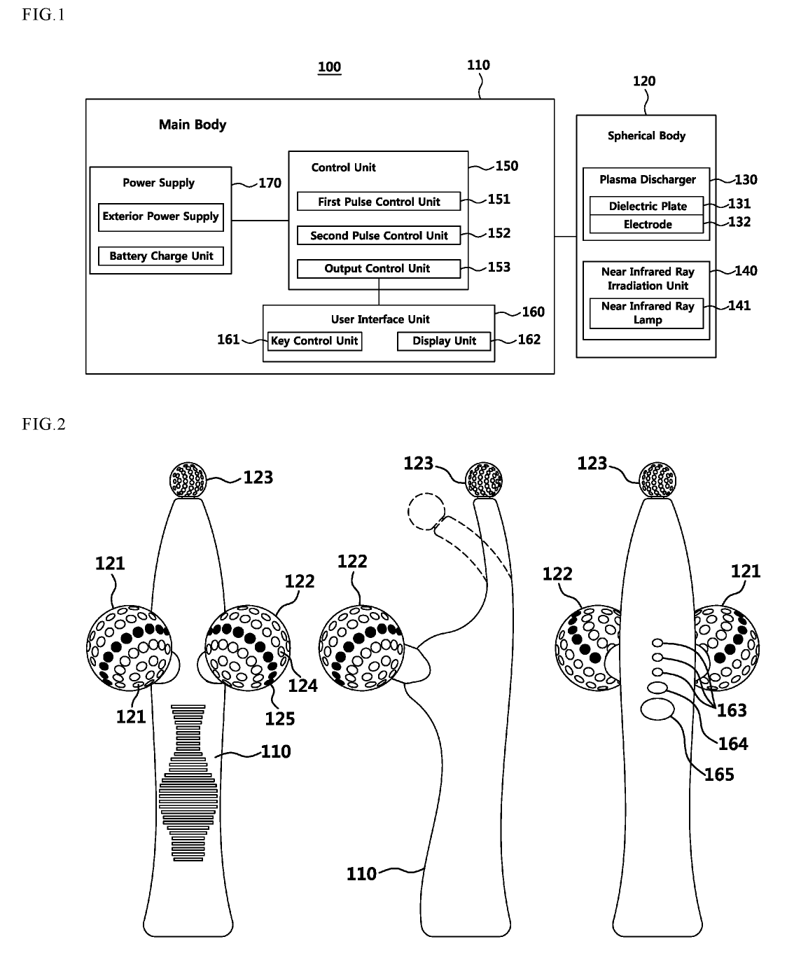

FIG. 1 is a diagram illustrating a skin care apparatus using plasma and near-infrared ray according to an exemplary embodiment of the present disclosure.

A skin care apparatus 100 using plasma and near-infrared ray according to an exemplary embodiment of the present disclosure includes dual electric generation of plasma and near-infrared ray source light.

A skin care apparatus 100 using plasma and near-infrared ray according to an exemplary embodiment of the present disclosure provides cosmetic procedures on an epidermal layer through a plasma electric generation in appropriately controlled and concentrated state using ionized materials in the air. Herein, the epidermal layer dignifies the region where cells are generated.

Further, a skin care apparatus 100 according to an exemplary embodiment of the present disclosure improves skin-tone and deactivates enzymes that cause skin damage while regenerating skin with the recovery of collagen damage as well as common cells by permeating the wave of light into the dermis layer through the light source of the near-infrared ray region. Herein, the dermis layer signifies where collagen and elastin are generated.

Thus, a skin care apparatus 100 according to an exemplary embodiment of the present disclosure is directed to providing effects of ameliorating wrinkles and lifting through a plasma skin ball including dual electric generation. The skin care apparatus 100 may be used as a home beauty care apparatus in the form of a face roller using a plasma skin ball.

As illustrated in FIG. 1, a skin care apparatus 100 using plasma and near-infrared ray according to an exemplary embodiment of the present disclosure includes a main body 110, a spherical body 120, a plasma discharger 130, a near-infrared irradiation unit 140, a control unit 150, a user interface unit 160, and power supply 170.

Each component of a skin care apparatus 100 using plasma and near-infrared ray of FIG. 1 will be described more fully hereinafter.

A main body 110 is in the form of handheld that allows a user to operate without connecting a cable. The main body 110 is linked to a spherical body 120 and takes the role of supporting the spherical body 120.

The spherical body 120 rotates in connection with a rotational axis of the main body 110. The spherical body 120 includes a plurality of plasma discharge grooves 124 formed around the surface of the spherical body 120 and a plurality of near-infrared irradiation grooves 125.

And the plasma discharger 130 includes a dielectric plate 131 and an electrode 132 that respectively correspond to the plasma discharge grooves 124. The plasma discharger 130 discharges plasma to the skin contacted by the rotating spherical body through barrier discharge caused by the dielectric plate 131 and the electrode 132.

Further, the near-infrared irradiation unit 140 includes a near-infrared light source connected with the near-infrared irradiation grooves 125. The near-infrared irradiation unit 140 irradiates near-infrared ray generated from the near-infrared light source to the skin contacted with the rotating spherical body 120 through the near-infrared irradiation grooves 125.

Hereinafter, a plasma electric generation and a near-infrared ray electric generation according to an exemplary embodiment of the present disclosure will be described.

The plasma discharger 130 transforms moisture and oxygen in the air into plasma status. The plasma discharger 130 efficiently and rapidly interfuses it into the skin, accelerates collagen, and helps the amelioration of wrinkles and the increase in skin elasticity.

The plasma discharger 130 brightens the skin tone by inhibiting melanin synthesis that subdues the skin. Also, plasma can help improve skin troubles by its sterilizing power similar to a high temperature of 3500°, more powerful than ultraviolet ray, and safely applying it to the skin.

To simply looking at the principle of plasma, the plasma discharger 130 generates micro plasma using ions in the air. And the plasma discharger 130 sublimates fibrous tissues of the outer layer.

The plasma can be applied to sterilizing effects, healing acceleration, stopping of bleeding, healing of wrinkles and skin hyper-pigmented diseases, dental bleaching, and treatment of cancer.

Meanwhile, near-infrared ray irradiated from the near-infrared irradiation unit 140 signifies light, the wavelength of which is between 760˜1500 nm. Among the wavelengths, the wavelength 630 nm is harmless to human body and is able to treat a large area of affected body by appropriate output of light, distinguished from a high-powered laser that intensively treats a localized region.

Herein, the near-infrared light source works by inducing an optical-biochemical response between cells. The near-infrared ray may be medically applied to wound healing, infection healing, skin reproduction, prevention of burn, and photodynamic therapy.

Meanwhile, a power supply 170 supplies power to each component of the skin care apparatus 100 in the form of handheld. The power supply 170 can store power needed for plasma discharge and near-infrared ray irradiation and supply it to each component.

The power supply 170 can be provided with necessary power for instance, an exterior power or a battery power. Further, the power supply 170 can provide the charging of a battery through charge circuit. Further, the skin care apparatus 100 may be used as an independent device, charged without the connection of electric wires or electric links. For instance, the power supply 170 can include an exterior power supply 171 or a battery charge unit 172.

A user interface unit 160 receives a user input for plasma discharge or near-infrared ray irradiation. The user interface unit 160 receives the input of set-up orders of plasma discharge and near-infrared ray irradiation or manipulation orders for instance, power on and off, the change of mode from users.

And the user interface unit 160 demonstrates the results of user inputs. The user interface unit 160 can demonstrate operation information regarding plasma discharge and near-infrared ray irradiation for instance, plasma discharge and the status of near-infrared ray irradiation, the status of output to users. Further, the user interface unit 160 notices the non-contact information to users according to the controls of a control unit 150.

As illustrated in FIG. 1, the user interface unit 160 may include a key control unit 161 and a display unit 162. The key control unit 161 can establish a system through a user's input of a key or can perform setting such as a near-infrared ray level and a plasma output. Further, the display unit 162 can display the status of a system or the result of a set-up according to such key inputs. The display unit 162 allows users to check the strength of plasma output and the operation status of the apparatus by displaying them on the equipped Led indicator.

Meanwhile, the control unit 150 controls the plasma discharger 130 and the near-infrared irradiation unit 140. Accordingly, the control unit 150 controls plasma discharge at the plasma discharger 130 and near-infrared ray irradiation at the near-infrared irradiation unit 140. The control unit 150 controls the pulses and output of plasma and near-infrared ray according to the pulse pattern or output pattern established for a user skin type. Herein, the pulse pattern may be a pattern generated by combining the interval or the cycle of plasma pulse and near-infrared ray pulse according to a pre-established condition. The output pattern may be a pattern generated by combining weak output, medium output, and strong output according to a pre-established condition.

As illustrated in FIG. 1, the control unit 150 may include a first pulse control unit 151, a second pulse control unit 152 and an output control unit 153. The control unit 150 is an embedded processing unit into the skin care apparatus 100 and performs the role of controlling the entire action.

The first pulse control unit 151 controls plasma pulses according to the pulse patterns established for a user skin type and controls plasma discharged from the plasma discharger 130.

Further, the second pulse control unit 152 controls near-infrared ray pulses according to the pulse patterns established for a user skin type and controls the near-infrared ray pulses irradiated from the near-infrared irradiation unit 140.

The first and the second pulse control units 151, 152 controls plasma pulses and near-infrared ray pulses according to various algorithms suitable for a user skin type by precise current control through PWM (Pulse Width Modulation).

The output control unit 153 controls the output of plasma and near-infrared ray according to output patterns established suitable for a user skin type. The output control unit 153 controls plasma and near-infrared ray outputs. The output control unit 153 can establish stages of the output of plasma and near-infrared ray to improve the adaptability of the skin. This is for the purpose of adapting to the various skin types of users. The output control unit 153 can generate output patterns suitable for a user's environment by providing various forms of output.

For instance, the control unit 150 can control the plasma discharger 130 and the near-infrared irradiation unit 140 to apply the pattern function of plasma electric stimulation the structure of wave. The control unit 150 can control the plasma discharger 130 and the near-infrared irradiation unit 140 according to various applied algorithms by output control of plasma.

Thus, the control unit 150 can generate various current stimulating patterns suitable for various skin thicknesses and senses of the users. For instance, the control unit 150 gradually increases the output of plasma or near-infrared ray and controls the output through a set-up input at the user interface unit 160 the moment when the output arrives at a level that a user thinks appropriate.

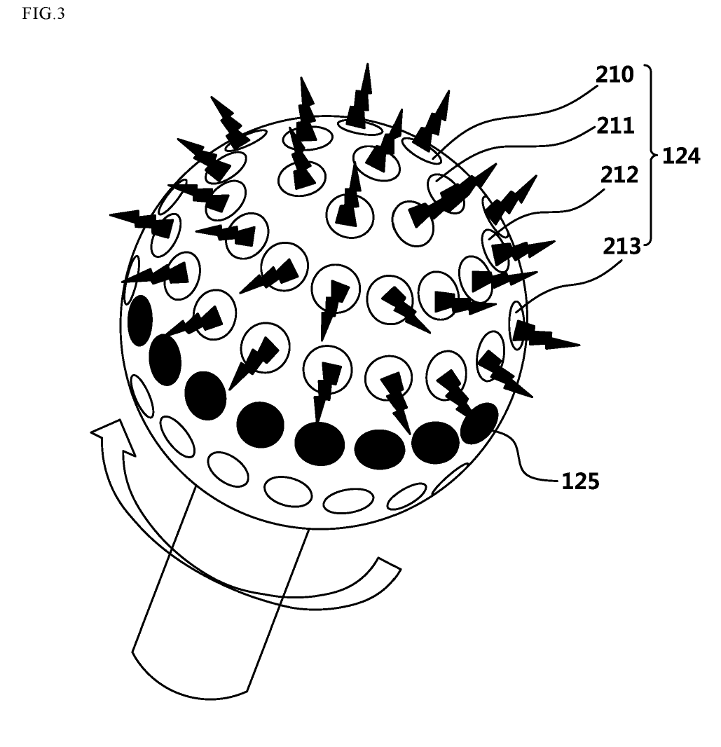

FIG. 2 is a drawing illustrating the lower side, the side, and the upper side of a skin care apparatus using plasma and near-infrared ray.

The skin care apparatus 100 according to an exemplary embodiment of the present disclosure irradiates a dual electric generation of plasma and near-infrared ray on the rotating ball shape. Herein, the spherical bodies 121 to 123 may be called plasma skin balls.

The dielectric plate 131 and the electrode 132 equipped in the plasma discharger 130 generates plasma through a dielectric barrier discharge within the rotating plasma skin ball. Herein, the dielectric plate 131 is used for the dielectric barrier discharge, and the dielectric barrier discharge can prevent the skin thermal damage.

Herein, the skin contact angle of the spherical body the plasma skin ball is ergonomically designed lest plasma and near-infrared ray generate in the same region at the same time. This is to efficiently improve the skin.

As illustrated in FIG. 2, a spherical body 120 includes at least one pair of spherical bodies 121 and 122. At least one pair of spherical bodies 121 and 122 is connected to the axis of rotation of the main body 110. Herein, a support unit of the spherical body 120 connected to the axis of the rotation of the main body 110 may be composed of elastic material that allows angle control or articulated structure.

Meanwhile, the spherical body 120 may further include at least one more other spherical body 123 that is located between the at least one pair of spherical bodies 121 and 122. Herein, the other spherical body 123 is able to rotate in connection with the rotational axis of the main body and a plurality of plasma discharge grooves 124 and a plurality of near-infrared irradiation grooves 125 are formed on the other spherical body 123.

Hitherto, the spherical body 120 including one pair of spherical bodies 121 and 122 and the other spherical body 123 between them has been explained with reference to the FIG. 2. As those skilled in the art would realize, the described embodiments may be modified in various different ways, all without departing from the spirit or scope of the present disclosure.

For instance, the spherical body 120 of the FIG. 2 may include another pair of spherical bodies and another spherical bodies besides the one pair of spherical bodies 121 and 122 and the other spherical body between them 123.

For instance, although the spherical body 120 of FIG. 2 is explained as including the spherical body on which the plasma discharge groove 124 and the near-infrared irradiation groove 125 are formed, a plasma discharge groove 124 or a near-infrared irradiation groove 125 may be formed on each individual spherical body. Namely, it is possible that only a plasma discharge groove 124 is formed on a spherical body without a near-infrared irradiation groove 125, or that only a near-infrared irradiation groove 125 is formed without a plasma discharge groove 124.

Meanwhile, the main body 110 includes a mode display LED 163 that displays modes, a mode button 164 that selects modes, and a power button 165 that turns on or turns off the power.

FIG. 3 is a drawing illustrating the structure of plasma discharge and near-infrared ray irradiation according to an exemplary embodiment of the present disclosure.

The spherical body 120 illustrated in FIG. 2 includes at least one pair of spherical bodies 121 and 122 on which a plurality of plasma discharge grooves 124 and near-infrared irradiation grooves 125 are formed as illustrated in FIG. 3.

A plurality of plasma discharge grooves 124 that respectively have a pre-established depth according to the generating condition of plasma are formed on each surface of the at least one pair of spherical bodies.

To more closely describe a plurality of plasma discharge grooves 124, a plurality of plasma discharge grooves from 210 to 213 that are divided by a plurality of border layers are formed on the surface of the spherical body.

The FIG. 3 illustrates plasma discharge grooves with boundary layers from 1 to 3 211 to 213 and a peak 210 of a plasma discharge groove 124 on the upper part of the spherical body. Herein, although the boundary layer of plasma discharge groove is in the circular form, it is not restricted to a certain location or a certain shape of the surface of the spherical body. Further, although the boundary layers of plasma discharge grooves are from 1 to 3 211 to 213, it is also possible that a plasma discharge groove 124 may be formed with various numbers and locations.

For instance, the control unit 150 may control the discharge of the plasma discharger 130 through a boundary layer set for a user's skin type among the boundary layers of plasma discharge grooves from 1 to 3 211 to 213.

For another example, the control unit 150 may control the plasma discharger 130 so that plasma discharge may occur through the plasma discharge groove 124 located at regular spaces for instance, an odd numbered plasma discharge groove or an even numbered plasma discharge groove.

For another example, the control unit 150 may control the near-infrared irradiation unit 140 so that near-infrared ray irradiation may occur through a near-infrared irradiation groove 125 located at regular spaces for instance, an odd numbered near-infrared irradiation groove or an even numbered near-infrared irradiation groove.

The plasma discharger 130 allows the plasma skin ball to treat skin wrinkles in the way that it ionizes materials in the air and generates energy when the contact surface of the plasma skin ball contacts with the skin. Herein, the spherical body of the plasma skin ball may be a face roller shape of a user.

Further, the near-infrared light source for instance, 630 nm is absorbed in the fibroblast in the capillary, promotes skin activation, and regenerates skin. The near-infrared light source enables skin treatment without causing skin damage as it does not generate heat and only irradiates light energy.

For instance, when a user treats the facial skin, the skin care apparatus 100 enables the dual electric generation of plasma and near-infrared ray at the spherical body from 121 to 123, which is in the shape of a rotating ball, for face lifting.

The skin care apparatus 100 may prevent the heat damage of skin caused by plasma electric generation as the ceramic dielectric plate 131 is located inside the rotating spherical body from 121 to 123.

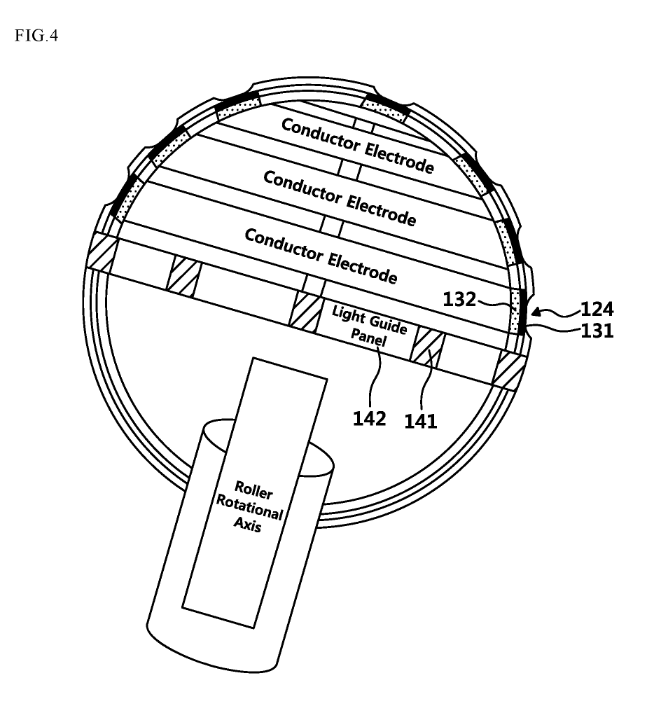

FIG. 4 is an end view of the inside of the spherical body according to an exemplary embodiment of the present disclosure.

As illustrated in FIG. 4, the plasma discharger 130 is equipped with a dielectric plate 131 and an electrode 132.

Herein, the dielectric plate 131 is located corresponding to the plasma discharge groove 124 formed on the surface of the spherical body. Further, the electrode 132 is linked with the dielectric plate 131 and is installed inside the spherical body.

The plasma discharge groove 124 is formed on the surface of the spherical body that is divided by a plurality of boundary layers. Accordingly, the electrode 132 is installed as an electrode boundary at a boundary layer of each plasma discharge groove. At this moment, the whole electrode boundaries are linked with the main electrode in the center.

For modified example, each electrode boundary can individually turn on or off power according to the control of the control unit 150.

The near-infrared irradiation unit 140 is equipped with a near-infrared ray lamp 141, a near-infrared light source, and a light guide panel 142.

Herein, the near-infrared ray lamp 141 generates near-infrared ray. And the generated near-infrared ray is conveyed to each near-infrared irradiation groove 125 through the light guide panel 142 and is irradiated through the near-infrared irradiation grooves 125.

FIG. 5 is a structure drawing illustrating the dielectric plate of the plasma discharger and the electrode according to an exemplary embodiment of the present disclosure.

As illustrated in FIG. 5, the dielectric plate 131 and the electrode 132 of the plasma discharger 130 are located inside of at least one pair of spherical bodies of the spherical body 120.

The spherical body is linked to the rotational axis of the main body 110 and rotates when a user moves the skin care apparatus 100 by contacting it to the skin 101. Examining the skin 101 contacted by the rotating spherical body 120, the dielectric plate 131 and the electrode 132 of the plasma discharger 130 generate dielectric barrier discharge according to the control of the control unit 150 by the current supplied by the main electrode 132.

And the plasma generated by dielectric barrier discharge is discharged on the skin of a user contacted by the spherical body.

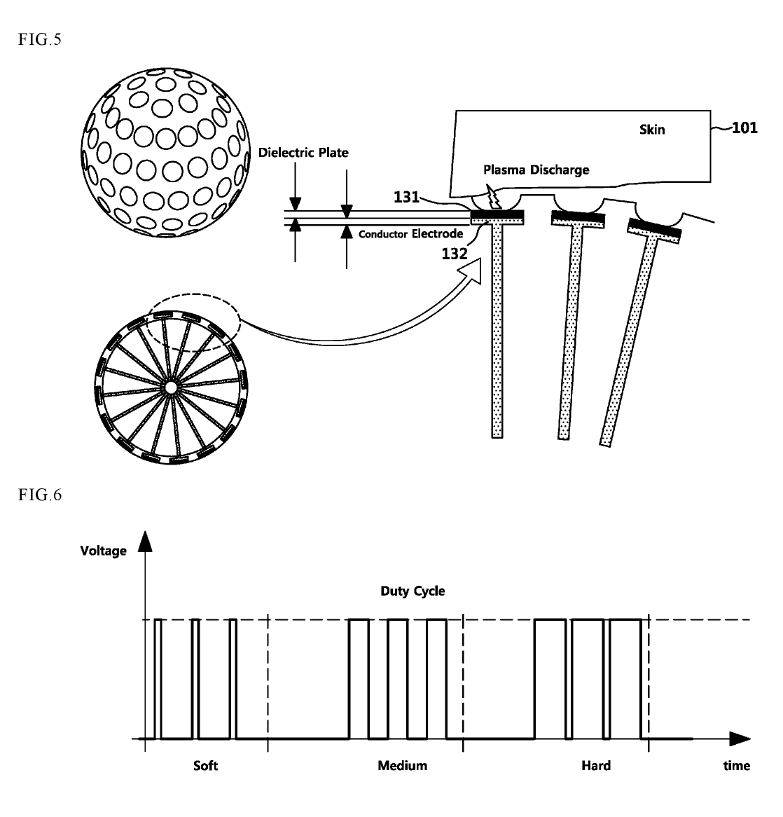

FIG. 6 is a graph illustrating pulse width modulation control waveform controlled by the control unit according to an exemplary embodiment of the present disclosure.

As illustrated in FIG. 6, the control unit 150 performs precise electric current control through PWM (pulse width modulation).

For instance, the control unit 150 controls the current by three-level pulse width modulation from soft, medium, to hard. The control unit 150 controls plasma pulse or near-infrared ray pulse by dividing the pulse width into three levels.

The control unit 150 performs various algorithms suitable for a user's skin type while accurately controlling the current through the pulse width modulation.

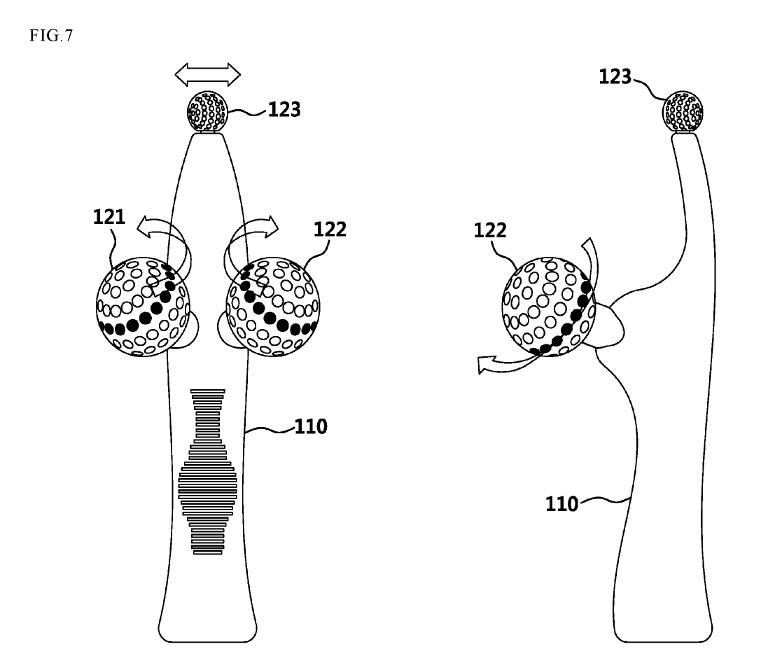

FIG. 7 is a diagram illustrating the roller action of a skin care apparatus using plasma and near-infrared ray according to an exemplary embodiment of the present disclosure.

As illustrated in FIG. 7, the skin care apparatus 100 using plasma and near-infrared ray according to an exemplary embodiment of the present disclosure enables a user to contact the spherical body 121 to 123 to the skin when the user grabs the main body 110.

When the spherical body moves in contact with a user's skin, one pair of spherical bodies rotate around in contact with the user's skin as illustrated in FIG. 7.

Accordingly, the skin care apparatus 100 performs plasma discharge and near-infrared ray irradiation on the region of absorption massage by the rolling of two spherical bodies.

For instance, the other spherical body can be used in the narrow region such as eyelids, eye rims, nasolabial lines, and the corner of mouth. Also, three spherical bodies can be used at the same time.

The connection part of the spherical body 120 linked with the rotational axis of the main body 110 is connected in the form of an arm that is capable of angle control. For instance, such connection part can be elastic material or articulated structure.

Meanwhile, a skin care apparatus 100 according to an exemplary embodiment of the present disclosure may double the clinical effects caused by plasma.

The skin care apparatus 100 according to an exemplary embodiment of the present disclosure can generate massage effects by two spherical bodies and plasma and near-infrared ray on a user's skin. Further, the skin care apparatus 100 according to an exemplary embodiment of the present disclosure can perform the same function in narrow regions as well as in wide regions through three spherical bodies.

The skin care apparatus 100 according to an exemplary embodiment of the present disclosure prevents dual irradiation caused by rolling as it is formed in the shape of spot contact type spherical body for both plasma discharge groove 124 and near-infrared irradiation groove 125 respectively.

Meanwhile, the operation procedure of the skin care apparatus 100 according to an exemplary embodiment of the present disclosure is as follows:

First, the skin care apparatus 100 performs product preparation for plasma discharge when a user turns on the power.

The skin care apparatus 100 performs plasma discharge through a photo sensor that reacts to the skin contact. At the moment, the power of the plasma discharger 130 goes off when it is not in contact with the skin of a user.

The plasma discharge cycle can be appointed for gentle rubbing. The plasma discharger 130 infiltrates into the molecular units inside the skin by ions, different from intense heat energy of the conventional laser, and performs collagen regeneration by dismantling keratinocytes, sterilization, and stimulating fibroblast.

For instance, the skin care apparatus 100 automatically turns the power off after ten minutes in order to restrict excessive use. In terms of the power off function, one time use may be a standard.

After using the apparatus, the cleaning of contamination caused by sebaceous gland foreign substance contact and the storage follow.

Meanwhile, the near-infrared ray will be explained hereinafter.

In terms of near-infrared ray, the near-infrared irradiation unit 140 operates at the same time when plasma discharge operates by the contact with the skin.

The near-infrared ray promotes blood circulation and nutrition supply to the skin as the red color wavelength of 630 nm is absorbed into the fibroblast and accelerates the skin metabolism.

At this moment, when the plasma discharge action turns off, the near-infrared ray irradiation action turns accordingly off.

The spherical body in the form of roller designed to lift the skin is a plasma skin-ball using dual electric generation and can maximize the effects of the amelioration of wrinkles and the skin lifting.

Meanwhile, the skin care apparatus 100 according to an exemplary embodiment of the present disclosure can be used as a home beauty-care apparatus, as an apparatus that improves wrinkles and skin flexibility. Further, the skin care apparatus 100 can be used as an apparatus that helps improvement of the skin.

Thus, the skin care apparatus 100 according to an exemplary embodiment of the present disclosure can improve wrinkles by using a dual electric generation of plasma and near-infrared ray and maximizes skin lifting effects by using a spherical body in the shape of face roller.

While this disclosure has been described in connection with what is presently considered to be practical exemplary embodiments, it is to be understood that the disclosure is not limited to the disclosed embodiments, but, on the contrary, is intended to cover various modifications and equivalent arrangements included within the spirit and scope of the appended claims. Accordingly, the scope of the present disclosure shall be determined only according to the attached claims.

What is claimed is:

1. A skin care apparatus using plasma and near-infrared ray, comprising:
a main body in the form of handheld;
a spherical body connected with a rotational axis of the main body to be rotatable, the spherical body having a plurality of plasma discharge grooves and a plurality of near-infrared irradiation grooves formed on a surface thereof;
a plasma discharger configured to comprise a dielectric plate and an electrode respectively corresponding to the plasma discharge grooves and discharge plasma generated through dielectric barrier discharge caused by a current supplied to the dielectric plate and electrode on skin contacted with the spherical body being rotating;
a near-infrared irradiation unit configured to comprise a near-infrared light source linked with the near-infrared irradiation groove and irradiate a near-infrared ray generated from the near-infrared light source through the near-infrared irradiation groove on the skin contacted by the spherical body being rotating;
a control unit configured to control a plasma discharge at the plasma discharger and an irradiation of near-infrared ray at the near-infrared irradiation unit; and
a power supply configured to supply power.

2. The skin care apparatus of claim 1, wherein the spherical body comprises at least one pair of spherical bodies having a plurality of plasma discharge grooves formed on the surface thereof, the plurality of plasma discharge grooves having a depth conforming to the predetermined generation condition of plasma.

3. The skin care apparatus of claim 2, wherein the spherical body further comprises at least one other spherical body, being located between the at least one pair of spherical bodies, connected with the rotational axis of the main body to be rotatable, and has a plurality of plasma discharge grooves and a plurality of near-infrared irradiation grooves formed thereon.

4. The skin care apparatus of claim 1, wherein the surface of the spherical body is divided into a plurality of boundary layers and a plurality of plasma discharge grooves being formed on each of the boundary layers.

5. The skin care apparatus of claim 1, wherein a part of the spherical body connected to the rotational axis of the spherical body is made of an angle adjustable elastic material or by an articulated structure.

6. The skin care apparatus of claim 1, wherein the control unit controls a plasma pulse and near-infrared ray pulse or a output of plasma and near-infrared ray according to a pulse pattern or output pattern established for a user skin type.

7. The skin care apparatus of claim 6, wherein the control unit comprises:
a plasma pulse control unit configured to control the plasma pulse according to the pulse pattern established for the user skin type;
a near-infrared pulse control unit configured to control near-infrared ray pulse according to the pulse pattern established for the user skin type; and
a plasma and near-infrared ray output control unit configured to control the output of plasma and near-infrared ray according to the output pattern established for the user skin type.

8. The skin care apparatus of claim 1, further comprising a user interface unit configured to receive a user input for plasma discharge or near-infrared ray irradiation and display the results of the user input.
